(19) United States
(12) Patent Application Publication
(10) Pub. No.: US 2005/0090549 A1
(43) Pub. Date: Apr. 28, 2005

(54) Intrathecal gabapentin for treatment of pain

(75) Inventors: Keith R. Hildebrand, Houlton, WI (US); Linda M. Page, Woodbury, MN (US); Deanna S. Lane, Columbia Heights, MN (US); Dennis D. Elsberry, Plymouth, MN (US); David A. Clarahan, Blaine, MN (US); Jayantha H. Ratnayake, Blaine, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/807,828

(22) Filed: Mar. 24, 2004

Related U.S. Application Data
(60) Provisional application No. 60/513,681, filed on Oct. 23, 2003.
(60) Provisional application No. 60/513,682, filed on Oct. 23, 2003.

(51) Int. Cl. A61K 31/195; A61M 31/00

(52) U.S. Cl. 514/561; 604/500

(57) ABSTRACT

Methods for treating pain by administering gabapentin to cerebrospinal fluid of a patient are discussed. Compositions, particularly injectable compositions, containing gabapentin are also discussed. In addition, systems including an implantable pump having a reservoir for housing a composition, a catheter having a proximal portion coupled to the pump and having a distal portion adapted for administrating a composition to a cerebrospinal fluid of a patient, and a composition containing gabapentin, which composition is housed in the reservoir of the pump, is also discussed.

### RELATED APPLICATIONS

This application claims priority to Provisional Application Ser. No. 60/513,682, entitled “INJECTABLE GABAPENTIN COMPOSITIONS”, filed Oct. 23, 2003, and Provisional Application Ser. No. 60/513,681, entitled “INTRATHECAL GABAPENTIN FOR TREATMENT OF PAIN AND EPILEPSY”, filed on Oct. 23, 2003, which provisional applications are herein incorporated by reference in their entirety.

### FIELD OF THE INVENTION

This invention relates to medical devices, therapeutic methods, and compositions for delivering gabapentin to a patient.

### BACKGROUND

Chronic intractable pain is often difficult to treat. Opioid drugs such as morphine and hydromorphone, which are currently infused into the subarachnoid space around the spinal cord for the treatment of pain, have limited efficacy against neuropathic and mixed (nociceptive and neuropathic components) pain states. In addition, opioid monotherapy often leads to tolerance with increasing doses of intrathecal opioid needed to control the patient's pain. Two drugs (clonidine and bupivacaine) are currently used primarily in combination with opioids as a means of increasing the efficacy of intrathecal infusion against neuropathic pain conditions. Although these drugs are effective against neuropathic pain, they may be associated with significant side effects (clonidine: hypotension, bradycardia, sedation, and dry mouth; bupivacaine: motor weakness, paresthesia, numbness). In addition, at higher concentrations and dosages, bupivacaine may be neurotoxic. Moreover, neither clonidine nor bupivacaine is approved by the FDA for chronic intrathecal infusion.

Gabapentin is currently marketed as NEURONTIN in oral formulations only. It has been used primarily to treat epilepsy although it has been used off-label to treat neuropathic pain and has recently received an FDA-approval for the treatment of one type of neuropathic pain, post herpetic neuralgia. Although some gabapentin can access the CNS when administered orally, because gabapentin is transported across the gut and the blood-brain barrier via an active and saturable L-amino acid transporter, the amount of gabapentin reaching the CNS sites of action is limited. Because this transporter is saturable, even if the concentration of gabapentin in the plasma is increased, the amount which crosses the blood-brain barrier will remain constant. Because infusion of drugs directly into the intrathecal space bypasses the blood-brain barrier, higher levels of gabapentin in the CNS are achievable. This can be associated with greater efficacy and potentially less supraspinal side effects (sedation, dizziness). Preclinical studies in neuropathic pain models have demonstrated that bolus administration of gabapentin into the lumbar intrathecal space results in analgesic efficacy at doses much lower than required if gabapentin is administered systemically. Because the amount of gabapentin carried to the brain via the CSF is limited after intrathecal infusion, the supraspinal side effects of intrathecal gabapentin may be less than those associated with oral or systemic administration. Baclofen, a small molecule with a similar structure and side effect profile as gabapentin, produces significantly more sedation and dizziness when administered orally than via the lumbar intrathecal route. Although the analgesic properties of intrathecal gabapentin have been studied in preclinical models, these studies have only involved laboratory animal (rat and mice) pain models in which the drug was administered by bolus injection.

### SUMMARY OF THE INVENTION

An embodiment of the invention provides a system for delivering gabapentin to a cerebrospinal fluid of a patient to treat pain. The system comprises an amount of gabapentin effective to treat a pain when administered to a cerebrospinal fluid of a patient, an implantable pump housing the gabapentin, and a catheter coupled to the pump and adapted to deliver the gabapentin to a cerebrospinal fluid of the patient.

An embodiment of the invention provides a method for treating a pain in a patient in need thereof. The method comprises administering gabapentin to cerebrospinal fluid of the patient by way of an implantable pump system. In an embodiment, the pain is chronic intractable pain. In an embodiment the gabapentin is administered to the cerebrospinal fluid by infusing gabapentin into the subarachnoid space around the spinal cord.

Advantages of embodiments of the invention include greater control of CNS concentrations of gabapentin, improved efficacy of gabapentin for treatment of pain, and potential for reduced side effects relative to oral gabapentin. These and other advantages of the invention will become evident upon reading the description herein.

### BRIEF SUMMARY OF THE DRAWINGS

FIG. 1 is a diagrammatic illustration of a patient's brain, the associated spaces containing cerebrospinal fluid, and the flow of cerebrospinal fluid in the subarachnoid space.

FIG. 2 is a diagrammatic illustration of a pump system for delivering a composition comprising a therapeutic agent according to an embodiment of the present invention.

FIG. 3 is a diagrammatic illustration of a catheter implanted in a patient according to an embodiment of the present invention.

FIG. 4 is a diagrammatic illustration of an implanted catheter and pump in accordance with an embodiment of the present invention.

FIG. 5 is a diagrammatic illustration of a catheter and external pump in accordance with an embodiment of the present invention.

The drawings are not necessarily to scale. Like numbers refer to like parts or steps throughout the drawings.

### DETAILED DESCRIPTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like are meant to include methods to alleviate, slow the progression, prevent, attenuate, or cure the treated disease.

Cerebrospinal Fluid

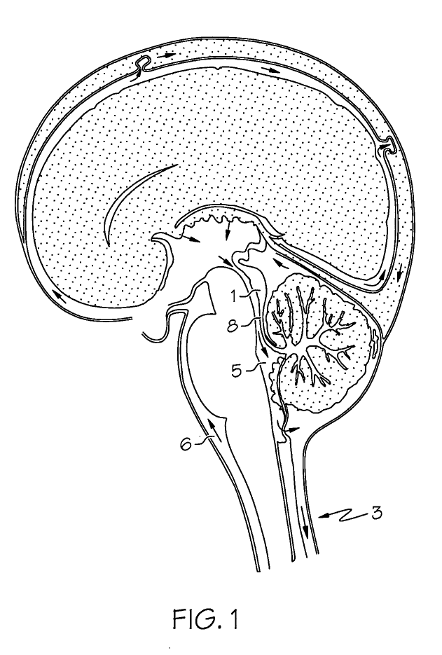

According to an embodiment of the invention, a composition comprising gabapentin may be delivered directly to cerebraspinal fluid 6 of a patient. Referring to FIG. 1, cerebrospinal fluid (CSF) 6 exits the foramen of Magendie and Luschka to flow around the brainstem and cerebellum. The arrows within the subarachnoid space 3 in FIG. 1 indicate cerebrospinal fluid 6 flow. The subarachnoid space 3 is a compartment within the central nervous system that contains cerebrospinal fluid 6. The cerebrospinal fluid 6 is produced in the ventricular system of the brain and communicates freely with the subarachnoid space 3 via the foramen of Magendie and Luschka. A composition comprising gabapentin may be delivered to cerebrospinal fluid 6 of a patient anywhere that the cerebrospinal fluid 6 is accessible.

According to an embodiment of the invention, a composition comprising gabapentin may be administered intrathecally to a patient. Intrathecal delivery of therapeutics into the cerebrospinal fluid 6 can be less invasive than intraparenchymal (direct tissue) delivery of therapeutics. In addition, intrathecal delivery of therapeutics may not require the need for a neurosurgeon as intrathecal delivery of therapeutics does not require delivery to a direct brain target. Other physicians may be qualified to insert a catheter into the subarachnoid space 3 of the spinal column in order to initiate intrathecal therapeutic delivery.

Delivery System

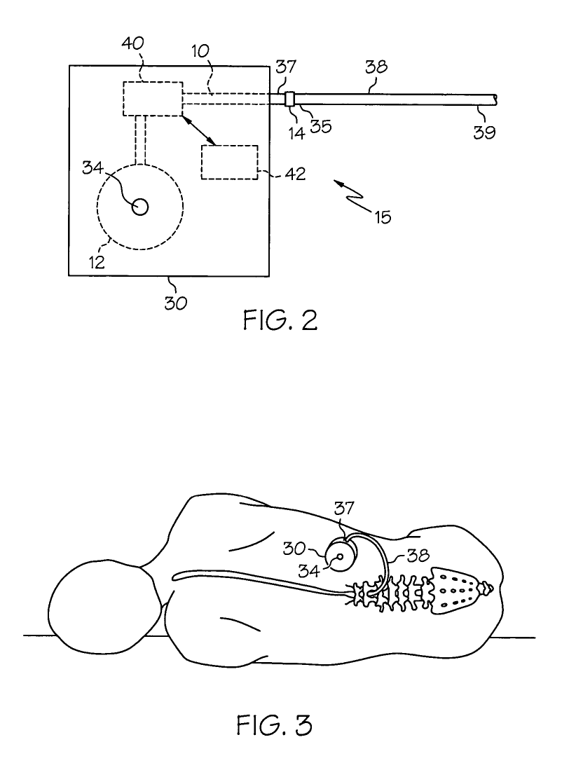

An embodiment of the invention provides a system for delivering to cerebrospinal fluid 6 of a patient a composition comprising gabapentin in an amount effective to treat pain in the patient. Referring to FIG. 2, a system 15 for delivering a composition comprising gabapentin is shown. The system 15 comprises a therapy delivery device 30. The device comprises a pump 40 coupled to a reservoir 12 for housing a composition comprising a therapeutic agent, such as gabapentin. The system 15 further comprises a catheter 38. The catheter 38 comprises a proximal portion 35 coupled to the pump 40 and a distal portion 39 adapted for infusing the composition to a patient's cerebrospinal fluid 6. It will be recognized that the catheter 38 may have one or more drug delivery regions along the length of the catheter 38 and that a drug delivery region may or may not be at the distal end 39 of the catheter 38. The therapy delivery device 30 may be implantable or may be an external device. The therapy delivery device 30 may have a port 34 into which a hypodermic needle can be inserted to inject a quantity of therapeutic agent into reservoir 12. The therapy delivery device 30 may have a catheter port 37, to which the proximal portion 35 of catheter 38 may be coupled. The catheter port 37 may be coupled to pump 40 through an internal catheter 10. A connector 14 may be used to couple the catheter 38 to the catheter port 37 of the device 30. Device 30 may take the form of the device shown in U.S. Pat. No. 4,692,147 (Duggan), assigned to Medtronic, Inc., Minneapolis, Minn., commercially available as the Synchromed® infusion pump, which is incorporated by reference.

The therapy delivery device 30, such as Medtronic's SYNCHROMED pump system, may be operated to discharge a predetermined dosage of the pumped fluid into the CSF 6 or brain of a patient. The therapy delivery device 30 may contain a microprocessor 42 or similar device that can be programmed to control the amount of fluid delivery. The programming may be accomplished with an external programmer/control unit via telemetry. A controlled amount of fluid comprising therapeutics may be delivered over a specified time period. With the use of a delivery device 30, different dosage regimens may be programmed for a particular patient. Additionally, different therapeutic dosages can be programmed for different combinations of fluid comprising therapeutics. Those skilled in the art will recognize that a programmed therapy delivery device 30 allows for starting conservatively with lower doses and adjusting to a more aggressive dosing scheme, if warranted, based on safety and efficacy factors.

If it is desirable to administer more than one therapeutic agent, the composition within the reservoir 12 may contain a second, third, fourth, etc. therapeutic agent. Alternatively, the therapy delivery device 30 may have more than one reservoir 12 for housing additional compositions comprising a therapeutic agent. When the device 30 has more than one reservoir 12, the pump 40 may draw fluid from the one or more reservoirs 12 and deliver the drawn fluid to the catheter 38. The device 30 may contain a valve coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the device 30. A catheter 38 may have more than one lumen. Each lumen may be adapted to deliver a therapeutic agent from one or more reservoirs 12 of the pump 40. It will also be understood that more than one implantable device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

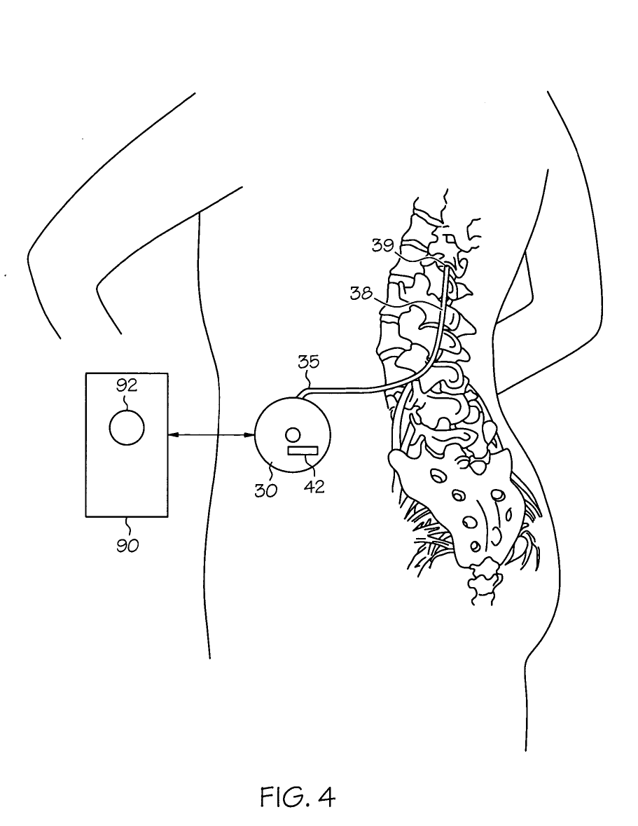

Referring to FIGS. 3 and 4, a device 30 may be implanted below the skin of a patient. Preferably, the device 30 is implanted in a location where implantation interferes as little as practicable with patient activity. Device 30 may be implanted subcutaneously in any medically acceptable area of the human body, such as in an abdominal pocket.

According to an embodiment of the invention, distal end 39 of the catheter 38 is positioned to infuse a fluid into a target area of a patient's CSF 6. As shown in FIG. 3, catheter 38 may be positioned so that the distal tip 39 of catheter 38 is located in the subarachnoid space 3 of the spinal cord between the fifth lumbar and fifth thoracic vertebrae. It will be understood that the distal tip 39 can be placed in a multitude of locations to deliver a therapeutic agent into the cerebrospinal fluid 6 of the patient. Within the spinal cord, the distal tip 39 of the catheter 38 may be inserted, for example, in the subarachnoid space 3 between the fifth thoracic (T5) and the first cervical vertebrae (C1), in the subarachnoid space 3 between the fifth lumbar (L5) and fifth thoracic vertebrae (T5), etc. The location of the distal tip 39 of the catheter 38 may be adjusted to improve therapeutic efficacy. The physician may also position the tip of the catheter to correspond to the level or dermatome of the patient's most significant pain or apparent origin of pain. As shown in FIG. 3, delivery of a composition comprising gabapentin into the CSF to treat pain can be accomplished by injecting the therapeutic agent via port 34 to catheter 38.

As shown in FIG. 4, a system for delivering therapeutic agent may include a patient-controlled activator 90, PCA. A PCA 90 may communicate with an implantable device 30 to adjust the amount of therapeutic agent delivered. Communication between PCA 90 and implantable pump 30 may be through any suitable means. In an embodiment, communication is through telemetry. Communication may be unidirectional; i.e., from PCA 90 to device 30, or bi-directional. PCA 90 may be a hand held device. PCA may contain a button 92 or other suitable means for a patient to indicate a desire to alter amount of therapeutic agent delivered. Typically, a patient will depress button 92 or activate other suitable means to direct device 30 to deliver additional therapeutic agent, such as a composition comprising gabapentin. Generally, a pulse or short-term increase in infusion rate of therapeutic agent will result as a result of the patient depressing the button 90. In an embodiment, a patient may place PCA 90 over skin in an area where device 30 is implanted. The amount and frequency of patient-controlled analgesic may be limited by a physician or other health care provider by specifically programming the PCA 90 for a particular patient. Preferably, such programming controls would be inaccessible to the patient. It will be appreciated that a similar PCA 90 feature can be included in an external pump without the requirement of an additional device component.

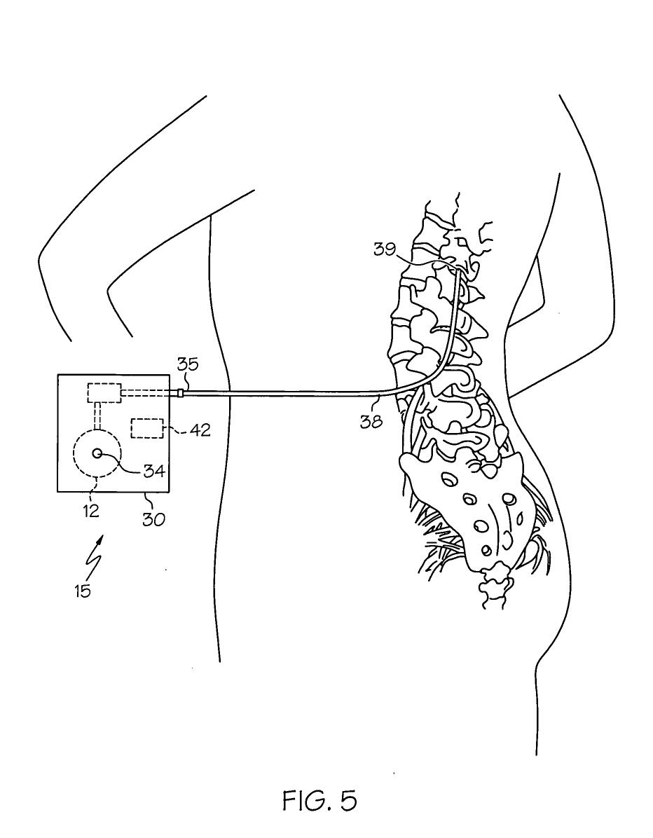

Referring to FIG. 5, a system having an external therapy delivery device 30 is shown. The proximal end 35 of a catheter 38 may be coupled to the device and the distal end 39 of the catheter 39 may be positioned to deliver a therapeutic agent pumped from the external device 30 through the catheter 38 to a patient's cerebral spinal fluid 6. As shown in FIG. 5, the therapeutic agent, such as gabapentin, may be administered intrathecally. External delivery device 30 may be used as part of a drug trial system prior to use of an implantable pump system, examples of which are shown in FIGS. 3 and 4. Use of an external delivery device 30 in such a manner provides an indication as to whether a patient will respond favorably to treatment prior to subjecting the patient to surgery associated with an implantable pump system. With a drug trial system, a catheter 38 may be placed to deliver a composition comprising a therapeutic agent epidurally to the patient. It will be recognized that the therapeutic agent may be administered directly to a patient's CSF 6 as discussed above. As with the implantable delivery devices (see FIGS. 3 and 4 and accompanying discussion), the placement position of the catheter may be varied from patient to patient or within a patient to optimize therapeutic efficacy. Any dose of therapeutic agent may be administered with an external therapy delivery device according to various embodiments of the invention. When used as a drug trial system, the dose of a therapeutic agent is typically started conservatively with lower doses and adjusted to higher doses until pain relief is noticed. It will also be recognized that single or multiple injections, without the use of a device 30, may also be used as to screen patients that are favorable candidates for an implantable therapy delivery device.

Treatment of Pain

An embodiment of the invention provides a method for treating pain. The method comprises delivering to cerebrospinal fluid 6 of a patient a composition comprising gabapentin in an amount effective to treat pain in the patient. The patient may be human. According to the International Association for the Study of Pain (IASP), “Pain is an unpleasant sensory and emotional experience associated with actual or potential tissue damage, or described in terms of such damage.” When pain is no longer associated with actual or potential tissue damage, it is considered chronic pain. The method may be effective in treating pain, whether acute, chronic or both. For treatment of chronic pain, a system comprising an implantable therapeutic pump 30 is preferably used.

With regard to chronic pain, an embodiment of the invention provides a method for treating nociceptive pain, neuropathic pain, or mixed pain (i.e., both nociceptive and neuropathic pain) by administering a composition comprising gabapentin to cerebrospinal fluid 6 of a patient in need thereof. Nociceptive pain, which originates in the viscera or limbs, is caused by actual or potential tissue damage and is conveyed to the brain via afferent pain fibers through the dorsal horn of the spinal cord. Afferent pain fibers enter the spinal cord at different locations depending on the origin of nociceptive pain. Thus, it may be desirable to deliver a composition comprising gabapentin to a spinal cord region associated with pain being sensed by a patient. For example, for low back and leg pain, gabapentin may be infused into the lumbar—low thoracic subarachnoid space. Additional antinociceptive agents (in addition to gabapentin) may also be administered. Any antinociceptive agent may be co-administered with gabapentin. Suitable antinociceptive agents include opioid agonists, non-steroidal anti-inflammatory drugs (NSAIDs), and GABA agonists such as baclofen. Exemplary opioid agonists include morphine and hydromorphone.

In an embodiment, the invention provides a method for treating neuropathic pain by administering a composition comprising gabapentin to cerebrospinal fluid 6 of a patient in need thereof. Any type of neuropathic pain may be treated according to the invention. Neuropathic pain can be caused by damage to the peripheral or central nervous system (nerve damage). Neuropathic pain as defined by IASP is: “pain initiated or caused by a primary lesion or dysfunction in the nervous system”. Classic examples of neuropathic pain include: trigeminal neuralgia, complex regional pain syndrome (CRPS), post herpetic neuralgia, diabetic neuropathy, and pain associated with plexopathy and radiculopathy. The etiology of neuropathic pain is typically classified according to the insult/injury to the nervous system or the anatomical distribution of the pain. It is generally classified as peripheral nerve injury such as polyneuropathy (e.g. diabetes, HIV, alcohol) and mononeuropathy or multiple mononeuropathy (e.g. diabetes, cancer, postherpetic neuralgia, ischemic neuropathy) as well as central nervous system injury (e.g. post stroke pain, spinal injury, multiple sclerosis). Regardless of the etiology, a method according to the invention may be used to treat neuropathic pain. It will be understood that the location of intrathecal delivery of a composition comprising gabapentin may be adjusted to an appropriate level of the spinal cord based on the origin of the neuropathy. Additional therapeutic agents (in addition to gabapentin) may also be administered to treat neuropathic pain. Any effective therapeutic agent may be co-administered with gabapentin including local anesthetics, alpha2-adrenergic agonists avd/or GABA agonists such as baclofen. Opioids and NSAIDS may be used, although neuropathic pain is considered less responsive to typical analgesics such as opioids and NSAIDs. Adjuvant analgesic agents, such as anticonvulsants and antidepressants may also be used.

In an embodiment, the invention provides a method for treating mixed pain in a patient by administering a composition comprising gabapentin to cerebrospinal fluid 6 of the patient. “Mixed pain” refers to pain that emerges from both nociceptive and neuropathic sources. Any mixed pain may be treated according to the invention. Exemplarily types of mixed pain that may be treated include chronic back and leg pain.

It will be understood that the amount of therapeutic agent delivered or the location of delivery of the therapeutic agent may be altered based upon the response of a patient to the therapeutic agent. Any measure of pain improvement or worsening may be used to evaluate whether a therapy modification may be appropriate. Such determinations can be readily made by, for example, a physician attending to the patient's care. In an embodiment, a Visual Analog Scale (VAS) is used to assess pain. The VAS is typically either a horizontal or vertical straight line; usually 10 cm in length with the descriptors of “least possible pain” or “no pain” on one end and “worst possible pain” on the other. The patient marks on the line where their pain level is at the present moment. The distance from the patient's mark to the end of the line is the measure of severity of the pain. The measurement is reproducible, as shown in the correlation coefficients between successive measurements. It is one of the most sensitive measurements of pain. The VAS is easy to administer and understand. It has been administered to children as young as 5 years of age and they were able to use the scale.

It will be understood that the amount of gabapentin delivered or the location in which gabapentin is delivered may be adjusted based upon the presentation and severity of side effects in a patient. Side effects may be recognizable by the patient, a physician attending to the care of the patient, other health care professionals, and the like. A physician or other health care professional may adjust therapy parameters based on side effects. Side effects which may be associated with gabapentin include: somnolence, dizziness, ataxia, fatigue, motor weakness, nausea and/or vomiting.

In an embodiment of the invention, a system for delivering gabapentin to the cerebrospinal fluid of a patient for the purposes of treating pain includes a component which allows the patient to increase the dose of gabapentin being administered by the implanted pump 40. This type of intervention is generally known as patient-controlled analgesia and has proven useful in treating “break-through” pain, episodic pain not well controlled by the baseline level of analgesic administration. Because pain is subjective in nature and varies with patient activity, the patient is often the most appropriate person to assess the level of analgesia and treat accordingly. In one embodiment, the additional system component includes a PCA 90 that can be activated by the patient and which interacts with the implanted drug pump via telemetry. When the patient experiences increased pain or is about to initiate an activity which will increase his level of pain, the patient activates the PCA 90 by depressing the appropriate button 92 and placing the PCA 90 over the skin where the pump is implanted. An additional amount (pulse or short-term increase in the infusion rate) of gabapentin or gabapentin-analgesic combination is then administered. In an embodiment, a similar PCA 90 feature is included in an external pump system without the requirement of an additional device component.

Compositions

In an embodiment, the invention provides a method comprising administering to cerebrospinal fluid 6 of a patient a composition comprising gabapentin. As used herein, gabapentin refers to 1-(aminomethyl)cyclohexane acetic acid and pharmaceutically acceptable salts, esters, solvates, hydrates, and polymorphs thereof. 1-(aminomethyl)cyclohexane acetic acid is a γ-aminobutyric acid (GABA) analogue with a molecular formula of C₉H₁₇NO₂ and a molecular weight of 171.24. 1-(aminomethyl)cyclohexane acetic acid is freely soluble in water and in both basic and acidic aqueous solutions. 1-(aminomethyl)cyclohexane acetic acid has a structure of:

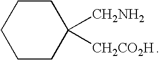

Gabapentin may be obtained from a variety of commercial sources, such as Shanghai Zhongxi International Trading Co., Shanghai, China; Hikal Limited, Bangalore, Karnaraka, India; Erregierre S.p.A., San Paolo d'Argon (BG), Italy; MediChem, SA, Sant Joan Despi (Barcelona), Spain; Ranbaxy Laboratories, New Delhi, India; Procos S.p.A., Cameri, Italy; Zambon Group, Milan, Italy; Hangzhuo Chiral Medicine Chemicals Co., Hangzhuo, China; InterChem Corporation USA, Paramus, N.J.; SST Corporation, Clifton, N.J.; Teva Pharmaceuticals USA, North Whales, Pa.; Plantex USA, Hakensack, N.J.; and Sigma-Aldrich, St. Louis, Mo., or an appropriate distributor. Alternatively, gabapentin may be synthesized and/or prepared as known in the art.

Any gabapentin composition suitable for administration to cerebrospinal fluid 6 may be used in a method according to the invention. Typically, the composition will be injectable. As used herein, “injectable composition” refers to a composition that is fluid at room temperature, which fluid is capable of being injected into a patient. Injectable compositions include solutions, suspensions, dispersions, and the like. Injectable solutions, suspensions, dispersions, and the like may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Solutions or suspensions comprising gabapentin may be prepared in water, saline, isotonic saline, phosphate-buffered saline, citrate-buffered saline, and the like and may optionally be mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient in which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption—for example, aluminum monosterate hydrogels and gelatin. Excipients that increase solubility, such as cyclodextrin, may be added.

Sterile injectable compositions may be prepared by incorporating a therapeutic agent in the desired amount in the appropriate solvent with various other ingredients as enumerated above and, as desired, followed by sterilization. Any means for sterilization may be used. For example, the injectable composition may be autoclaved, filter sterilized or heat treated following filter sterilization. In the case of sterile powders for the preparation of sterile injectable solutions, the preferred methods of preparation are vacuum drying and freeze-drying techniques, which yield a powder of the active ingredient plus any additional desired ingredient present in a previously sterile-filtered solution. Injectable compositions may be heat treated or sterilized by autoclaving.

Heat treatment, whether or not through autoclaving, may be performed at any combination of temperature and time necessary to sterilize a composition comprising gabapentin. For example, a composition may be subjected to heat treatment for about 2 minutes to about 60 minutes at temperatures of about 110° C. to about 140° C. Specific exemplary times and temperatures that may be used include 24 minutes at 121.1° C., 4 minutes at 130° C., 30 min at 118° C., and 6-8 min at 121.1° C. It will be recognized that with higher temperatures and the longer durations of heat treatment, the likelihood of gabapentin lactam formation will be increased. To prevent excess formation of lactam, the time and temperature of heat treatment may be adjusted to a combination that reduces lactam formation, yet continues to sterilize the composition comprising gabapentin.

In an embodiment, a composition comprising gabapentin is an injectable solution comprising an aqueous solvent. The solvent may be sterile water for injection or saline. The saline may be 0.9% (w/v) sodium chloride or a solution where just enough sodium chloride is added to make the final solution isotonic. The saline may be sterile saline. In an embodiment, the final solution has a pH between about 4 and about 9, between about 5 and about 7, between about 5.5 and abut 6.5, or about 6. The pH may be adjusted with HCl or NaOH. Preferably, the final solution contains less than about 5% of gabapentin lactam. In an embodiment, the final solution is essentially free of preservatives, buffers, or a combination thereof.

A composition comprising gabapentin according to an embodiment of the invention includes an amount of gabapentin effective to treat pain when administered to a patient's cerebrospinal fluid 6. When the composition is a solution or suspension, the gabapentin may be present in the composition at any concentration sufficient to treat pain. In an embodiment, gabapentin is present in a solution or suspension at a concentration between about 0.1 mg/mL and about 100 mg/mL. In an embodiment, gabapentin is present in a solution or suspension at a concentration between about 10 mg/mL and about 90 mg/mL. In an embodiment, gabapentin is present in a solution or suspension at a concentration between about 20 mg/mL and about 80 mg/mL. In an embodiment, gabapentin is present in a solution or suspension at a concentration of about 80 mg/mL. In an embodiment, a composition comprises between about 10 mg/ml and about 50 mg/ml gababentin. For example, the composition may comprise between about 20 mg/ml and 40 mg/ml, or about 30 mg/ml.

In an embodiment, an injectable composition comprising gabapentin is administered to cerebrospinal fluid 6 of a patient in a daily dose of between about 0.1 mg and about 200 mg. In an embodiment, gabapentin is administered in a daily dose of between about 1 mg and about 150 mg. In an embodiment, gabapentin is administered in a daily dose of between about 2 mg and about 60 mg. In an embodiment, gabapentin is administered in a daily dose of greater than about 25 mg. In an embodiment, gabapentin is administered in a daily dose of less than about 25 mg. For example, gabapentin may be administered at a daily dose of between about 0.1 mg and about 10 mg, between about 0.1 mg and 5 mg, between about 0.1 mg and 2 mg, between about 0.1 and 1 mg, between about 0.1 and 0.5 mg, or about 0.2 mg. It will be understood that daily dose requirements may be adjusted to account for variability in CSF volume, CSF production rates, and rate of clearance of gabapentin from the CSF. One of skill in the art will understand that such variability may be due in part to, e.g., gender and/or age. In an embodiment, the composition comprising gabapentin is administered intrathecally. An implantable therapy delivery device 30 may be used for intrathecal administration. When a therapy delivery device 30 is used, a composition comprising gabapentin may be infused into a patient's cerebrospinal fluid 6 through continuous infusion or as pulses over time. The rate of the infusion and the frequency and duration of the pulses may be controlled by a microprocessor 42 in the device 30.

A composition comprising gabapentin may be co-administered with one or more additional therapeutic agents for the treatment of pain. The one or more additional therapeutic agents may be administered in a separate composition from the composition comprising gabapentin, or the composition comprising gabapentin may further comprise one or more additional therapeutic agents. Preferably, the one or more additional theraputic agent is an analgesic or an adjuvant analgesic. Analgesic agents include opioids, NSAIDS, local anesthetics, and alpha2-adrenergic agonists. Adjuvent analgesics include anticonvulsants and antidepressants.

In an embodiment, a composition comprising gabapentin further comprises an opioid agonist. The opioid agonist may be, for example, morphine sulfate or hydromorphone HCl. Morphine and/or hydromorphone may be present in the composition at any concentration useful for treating pain. For example, morphine may be present in the composition at a concentration of between about 25 mg/mL and about 50 mg/mL. Hydromorphone may be present at a concentration of between about 1 mg/mL and about 20 mg/mL. It will be understood that the use of combination therapy may provide for increased efficacy while allowing for use of lower doses of each agent in the combination therapy (relative to if any agent were used alone in monotherapy). Decreased doses of each individual agent in combination therapy may limit side effects associated with any one of the individual agents. For example, combination therapy with gabapentin and opioids may allow for a decreased dose of an opioid. By decreasing opioid exposure, tolerance and dose escalation of the opioid can be reduced. In certain circumstances, it may be desirable to initiate therapy with a combination therapy rather than with monotherapy. For example, initiating combination therapy of gabapentin plus an opioid, instead of adding gabapentin to an ongoing opioid strategy in which significant tolerance has already developed, may be desirable.

In an embodiment, a composition comprising gabapentin further comprises a GABA agonist. The GABA agonist may be baclofen. A GABA agonist may be present in the composition at any concentration useful for treating pain. For example, baclofen may be present in the composition at a concentration of between about 10 and about 4000 mcg/ml, between about 50 and about 2000 mcg/ml, between about 1000 and about 4000 mcg/ml, and between about 20 and about 2000 mcg/ml. A GABA agonist, such as baclofen, may be administered at any daily dose effective for treating pain. Exemplary daily doses of baclofen include daily doses of between about 1 mcg and about 5 mg, between about 10 mcg and about 3 mg, and between about 50 mcg and about 2 mg. It will be understood that the use of combination therapy may provide for increased efficacy while allowing for use of lower doses of each agent in the combination therapy (relative to if any agent were used alone in monotherapy). Decreased doses of each individual agent in combination therapy may limit side effects associated with any one of the individual agents.

The following patent applications are generally relevant to injectable gabapentin and its use:
U.S. patent application Ser. No. ______, entitled INTRATHECAL GABAPENTIN FOR TREATMENT OF EPILEPSY, filed on even date herewith, and having Attorney Docket No. P-20905.00;
U.S. patent application Ser. No. ______, entitled INJECTABLE GABAPENTIN COMPOSITIONS, filed on even date herewith, and having Attorney Docket No. P-20904.00;
U.S. patent application Ser. No. ______, entitled PROCESS FOR PRODUCING INJECTABLE GABAPENTIN COMPOSITIONS, filed on even date herewith, and having Attorney Docket No. P-20907.00; and
U.S. patent application Ser. No. ______, entitled PUMP SYSTEMS INCLUDING INJECTABLE GABAPENTIN COMPOSITIONS, filed on even date herewith, and having Attorney Docket No. P-20906.00.

All patents, patent applications, technical papers, and other publications cited herein are hereby incorporated by reference herein, each in its respective entirety. As those of ordinary skill in the art will readily appreciate upon reading the description herein, at least some of the compositions, devices and methods disclosed in the patents and publications cited herein may be modified advantageously in accordance with the teachings of the present invention.

1. A system comprising:
a reservoir;
a pump coupled to the reservoir;
a catheter coupled to the pump and adapted for delivering a therapeutic agent to a cerebrospinal fluid of a patient; and
an injectable gabapentin composition housed in the reservoir and deliverable through the catheter in an amount effective to treat pain in the patient when administered to the cerebrospinal fluid of the patient.

2. The system of claim 1, wherein the amount of gabapentin administered is an amount effective to treat pain in a human.

3. The system of claim 1, wherein the injectable gabapentin composition is a solution.

4. The system of claim 3, wherein the solution comprises between about 0.1 mg/mL and about 100 mg/mL gabapentin.

5. The system of claim 4, wherein the solution comprises between about 10 mg/mL and about 80 mg/mL gabapentin.

6. The system of claim 5, wherein the solution comprises about 80 mg/mL gabapentin.

7. The system of claim 5, wherein the solution comprises between about 20 mg/ml and about 40 mg/ml gabapentin.

8. The system of claim 5, wherein the solution comprises about 30 mg/ml gabapentin.

9. The system of claim 3, wherein the solution comprises gabapentin and water.

10. The system of claim 9, wherein the solution comprises is sterile water.

11. The system of claim 9, wherein the solution further comprises sodium chloride.

12. The system of claim 11, wherein the solution comprises about 0.9% (w/v) sodium chloride.

13. The system of claim 11, wherein the solution comprises sodium chloride in an amount such that the solution is substantially isotonic with the cerebrospinal fluid.

14. The system of claim 11, wherein the solution comprises sterile saline.

15. The system of claim 9, wherein the solution has a pH between about 4 and about 9.

16. The system of claim 15, wherein the solution has a pH between about 5 and about 7.

17. The system of claim 16, wherein the solution has a pH between about 5.5 and about 6.5.

18. The system of claim 9, wherein the solution comprises essentially no preservatives.

19. The system of claim 9, wherein the solution comprises essentially no buffers.

20. The system of claim 3, wherein the solution comprises less than about 5% (w/v) of a gabapentin lactam.

21. The system of claim 6, wherein the solution has a pH between about 5.5 and about 6.5 and comprises essentially no preservatives, essentially no buffers, and less than about 5% (w/v) of gabapentin lactam.

22. The system of claim 1, wherein the pump is an implantable pump.

23. The system of claim 1, wherein the pump is an external pump.

24. The system of claim 1, wherein the injectable composition further comprises an opioid agonist.

25. The system of claim 1, wherein the injectable composition further comprises a GABA agonist.

26. The system of claim 25, wherein the GABA agonist is baclofen.

27. The system of claim 1, further comprising a patient controlled activator capable of instructing the pump to deliver an additional amount of the injectable composition.

28. A method for treating a pain in a patient in need thereof, the method comprising:
administering to a cerebrospinal fluid of the patient a composition comprising gabapentin in an amount effective to treat pain in the patient, wherein the gabapentin is administered by a system comprising a pump.

29. The method of claim 28, wherein the gabapentin is administered to the cerebrospinal fluid by infusing gabapentin into the subarachnoid space around the spinal cord.

30. The method of claim 28, wherein the pain is chronic pain.

31. The method of claim 30, wherein the chronic pain is nociceptive pain.

32. The method of claim 30, wherein the chronic pain is neuropathic pain.

33. The method of claim 30, wherein the chronic pain is mixed pain.

34. The method of claim 28, wherein gabapentin is administered at a daily dose of between about 0.1 mg and about 200 mg.

35. The method of claim 34, wherein gabapentin is administered at a daily dose of between about 1 mg and about 150 mg.

36. The method of claim 35, wherein gabapentin is administered at a daily dose of between about 2 mg and about 60 mg.

37. The method of claim 28, wherein gabapentin is administered at a daily dose of greater than about 25 mg.

38. The method of claim 28, wherein gabapentin is administered at a daily dose of less than about 25 mg.

39. The method of claim 38, wherein gabapentin is administered at a daily dose of between about 0.1 mg and about 10 mg.

40. The method of claim 28, wherein the pump is an implantable pump.

41. The method of claim 28, wherein the pump is an external pump.

42. The method of claim 28 further comprising administering an opioid agonist to the cerebrospinal fluid of the patient.

43. The method of claim 42, wherein the composition comprising gabapentin futher comprises the opioid agonist.

44. The method of claim 28 further comprising administering a GABA agonist to the cerebrospinal fluid of the patient.

45. The method of claim 44, wherein the GABA agonist is baclofen.

46. The method of claim 44, wherein the composition comprising gabapentin further comprises the GABA agonist.

47. The method of claim 28, wherein the patient controls the amount of the composition administered.

48. The method of claim 47, wherein the patient controls the amount of the composition administered by way of a patient-controlled activator.

49. The method of claim 47, wherein the patient controls the amount of the composition administered for treatment of episodic pain not well controlled by a baseline level of gabapentin administration.

50. A method for treating a pain in a patient in need thereof, the method comprising:
administering to a cerebrospinal fluid of the patient a composition comprising gabapentin in an amount effective to treat pain in the patient, wherein gabapentin is administered at a daily dose of greater than or equal to about 25 mg and wherein the patient experiences substantially no somnolence, dizziness, ataxia, or motor weakness due to the gabapentin.

51. A method for preparing a system of claim 1, comprising
adding the injectable gabapentin composition to the reservoir.
